(19) United States
(12) Patent Application Publication
(10) Pub. No.: US 2005/0278005 A1
(43) Pub. Date: Dec. 15, 2005

(54) Triggering plate of a chemical heating pack

(75) Inventor: Ching-Chuan Wang, Yung-Ho City (TW)

Correspondence Address: Dougherty & Troxell;ONE SKYLINE PLACE, SUITE 1404, 5205 LEESBURG PIKE, FALLS CHURCH, VA, 22041, US

(73) Assignee: Ten Comfor Corporation

(21) Appl. No.: 11/030,991

(22) Filed: Jan. 10, 2005

(30) Foreign Application Priority Data
Jun. 13, 2004 (TW) 093209464

(51) Int. Cl. A61F 7/00

(52) U.S. Cl. 607/96

(57) ABSTRACT

A triggering plate of a chemical thermos bag has at least a groove with a slit, having two opposite tip parts hiding each other and the tip parts being different in thickness.

### FIELD OF THE INVENTION

The present invention relates to a triggering plate (trigger) of a heating pack and particularly to a triggering plate with grooves being stamped to have oblique wall thickness thereof getting smaller due to special stamping tool (die).

### BACKGROUND OF THE INVENTION

A typical chemical heating pack is the cold/hot applying bag familiar to us and due to heat accumulation and releasing theory, a preset duration of cold or hot temperature can be maintained with the heating pack while it is applied to skin of human body for healing, health caring or physical curing. That is why the chemical heating pack can be seen in a family, a hospital and a clinic

In order to release heat, a triggering plate has to be placed in the heating pack for generating a fluctuation force to actuate the heat accumulating/releasing material such as ethylic acid sodium solution being crystallized and releasing heat.

Hence, the triggering plate design is an subject emphasized by the manufacturers and there are patents related the prior art such as Taiwanese Utility Model No. 80848, which was granted to the present inventor, entitled ELASTIC METAL SHEET IN A CHEMICAL heating pack and continuation applications for disclosing technique of the metal sheet.

Other prior art such as U.S. Pat. Nos. 4,077,390, 4,572,158, 4,872,442 and 5,205,278 has disclosed configuration and arrangement of the triggering plate.

FIG. 1 shows a top view of a conventional triggering plate and FIG. 2 shows a sectional view of the conventional triggering plate. The conventional triggering plate is made of metal mostly with a circular shape. One side of the conventional triggering plate is stamped with a plurality of grooves A and the grooves are disposed in a radial way symmetrically with respect to the center of the triggering plate. The grooves A are provided with identical wall thickness respectively, that is, the wall surrounding the respective groove A is the same thickness in spite of having local deformation.

Some of the preceding cited references of U.S. Patents have disclosed the metal sheet being stamped through vertically and this is one of the types frequently seen.

### SUMMARY OF THE INVENTION

A main object of the present invention is to provide a triggering plate of a chemical heating pack, which has at least a groove with a slit, having two opposite tip parts hiding each other and the tip parts being different in thickness.

### BRIEF DESCRIPTION OF THE DRAWINGS

The present invention can be more fully understood by reference to the following description and accompanying drawings, in which:

FIG. 1 is a top view of conventional triggering plate;

FIG. 2 is sectional view of the conventional triggering plate;

FIG. 3 is a sectional view of the triggering plate according to the present invention;

FIG. 4 is a perspective view of the triggering plate according to the present invention; and

FIG. 5 is a sectional view illustrating the triggering plate of the present invention being stirred as another moving direction.

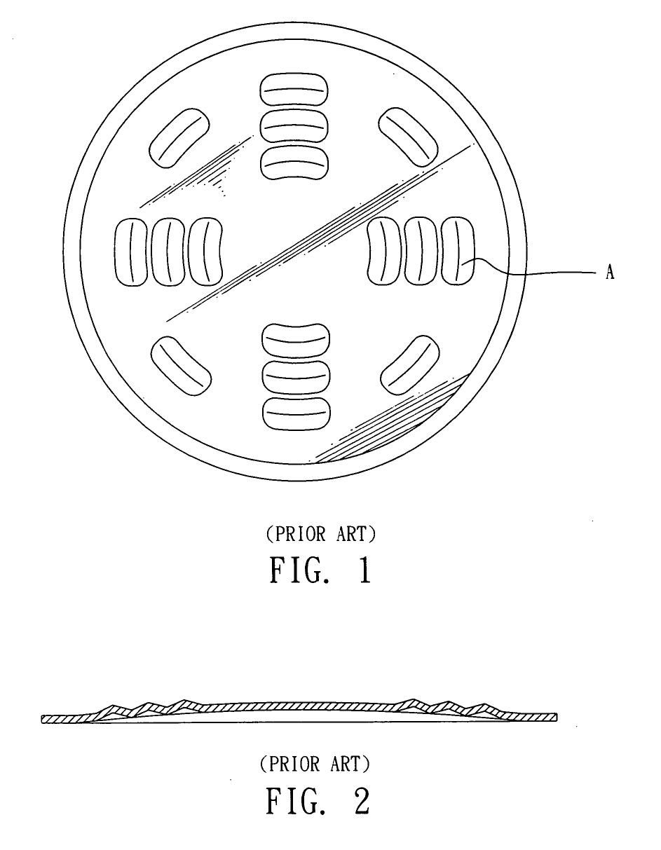

### DETAILED DESCRIPTION OF THE INVENTION

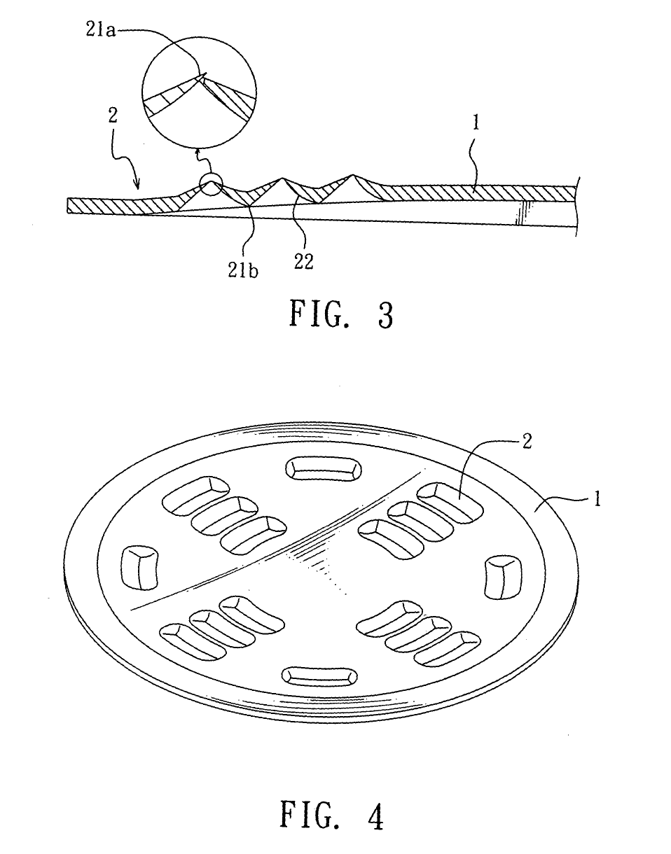

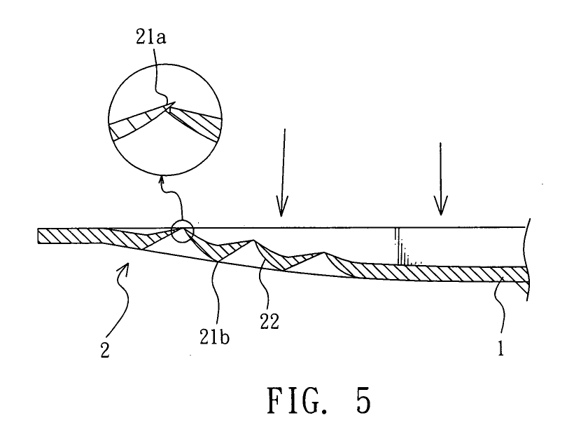

Referring to FIGS. 3, 4 and 5, basically, the improvement of the triggering plate according to the present invention resides in at least a groove formed with stamping so that the triggering plate 1 is provided with the groove 2. In case of two or more grooves being provided, the groves can be arranged in pairs and two grooves in each pair are disposed symmetrical to each other with respect to center of the triggering plate or the grooves can be arranged to be disposed continuously. Due to arrangement of the grooves being not the key point of the present invention, no detail will be described further. The triggering plate of the present invention is made of metallic material and the groove is obliquely formed by way of being stamped along cross-section of the triggering plate instead of being formed by way of being stamped vertically as the conventional triggering plate does. Further, the groove is provided with a little bit circular shape, that is, a side of the groove is concave downward and the other side thereof is concave upward.

Referring to FIG. 3 again, the groove 2 at the first side thereof has a thinnest tip 21a and at the second side thereof has a thickest base 21b with at least lateral circular recess 22. The tip 21a overlaps another tip 21a neighboring tip 21a during the circular moving direction being toward one side of the triggering plate and another tip 21a becomes overlapping the first tip 21a during the circular moving direction being toward another side of the trigging plate. In other words, the groove 2 is formed with stamping mold being provided with circular shapes and oblique moving direction such that the groove 2 is deformed with different thickness.

In practice, both sides of the groove 2 at the bottom thereof are different in thickness and opposite to each other and both tips 21a constitute interference each other with fluctuating force caused by the interference. Hence, it is helpful for starting heat accumulating/releasing material and it can supply fluctuating force to the conventional triggering plate with identical thickness.

It is appreciated that the present invention offers a problem-solving alternative for the triggering plate to break though puzzle thought of the groove being the same in thickness.

While the invention has been described with reference to the a preferred embodiment thereof, it is to be understood that modifications or variations may be easily made without departing from the spirit of this invention, which is defined by the appended claims.

1. A triggering plate of a chemical heating pack, comprising:
at least a groove with a slit, having two opposite tip parts hiding each other and the tip parts being different in thickness.

2. The triggering plate of a chemical heating pack as claimed in claim 1, wherein the groove has the bottom thereof being the thinnest.

3. The triggering plate of a chemical heating pack as claimed in claim 2, wherein the triggering plate between the bottom and the top thereof provides an oblique circular recess.

4. The triggering plate of a chemical heating pack as claimed in claim 1, wherein the triggering plate at both sides thereof has two or more continuous grooves respectively.

5. The triggering plate of a chemical heating pack as claimed in claim 4, wherein thickness of the grooves at one of the two sides are thin and that of the grooves at the other one of the two sides are thick.

6. The triggering plate of a chemical heating pack as claimed in claim 1, wherein the tip parts hide each other in a way of contact with each other.

7. The triggering plate of a chemical heating pack as claimed in claim 1, wherein the tip parts at both sides of the triggering plate are different in hiding due to stirring.

8. The triggering plate of a chemical heating pack as claimed in claim 1, wherein the triggering plate is made of metallic material.
